(19) United States
(12) Patent Application Publication
(10) Pub. No.: US 2011/0015383 A1
(43) Pub. Date: Jan. 20, 2011

(54) DERIVATIVES OF NUCLEOSIDE-5'-O-HYPOPHOSPHATES AND THEIR MONO- AND DITHIOHYPOPHOSPHATE ANALOGUES AND THE PROCESS FOR THE MANUFACTURE THEREOF

(76) Inventors: Wojciech J. Stec, Ksawerow (PL); Damian W. Blaziak, Konskie (PL)

Correspondence Address: BROOKS KUSHMAN P.C., 1000 TOWN CENTER, TWENTY-SECOND FLOOR, SOUTHFIELD, MI, 48075, US

(21) Appl. No.: 12/667,439

(22) PCT Filed: Jul. 1, 2008
(86) PCT No.: PCT/PL08/00049

§ 371 Date: Dec. 31, 2009

(30) Foreign Application Priority Data
Jul. 3, 2007 (PL) PL382824

(51) Int. Cl. C07H 19/20; C07H 19/10

(52) U.S. Cl. 536/26.7; 536/26.8

(57) ABSTRACT

The subject of the invention includes derivatives of nucleoside-5′-O-hypophosphates and their mono- and dithiohypophosphate analogues, in particular 5′-O-[β,β-dialkyl-(α-thiohypophosphate)]-, 5′-O-[β,β-dialkyl-(α,α-dithiohypophosphate)]-, 5′-O-[β,α-dialkyl-(α,β-dithiohypophosphate)-, 5′-O-[β-alkyl-(α-thiohypophosphate)]-, 5′-O-[β-alkyl-(α, oc-dithiohypophosphate)]-, 5′-O-(a-thiohypophosphate)]- and 5′-O-(a,a-dithiohypophosphate)nucleosides.

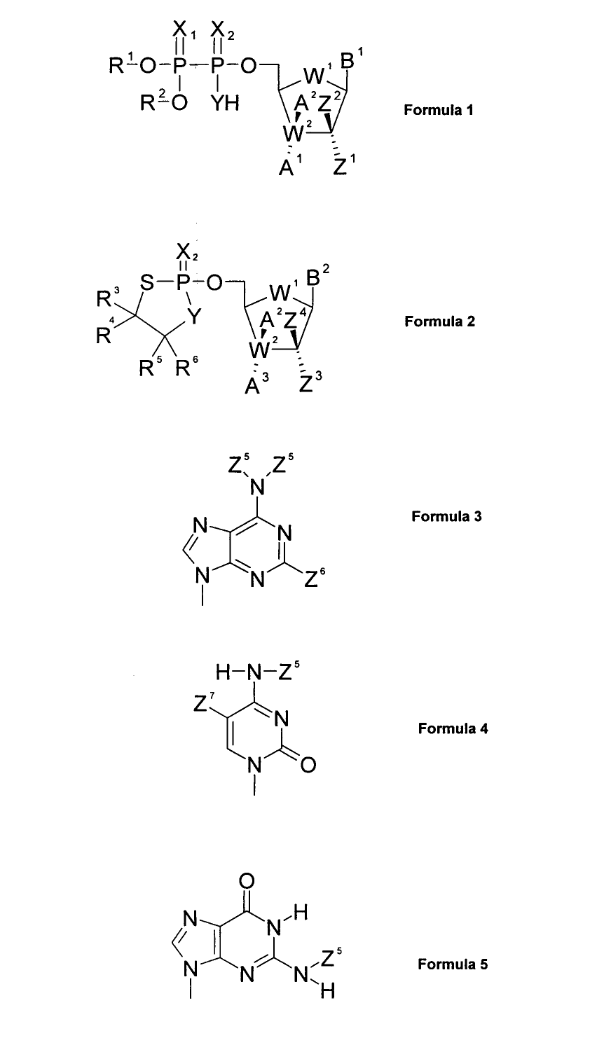

The subject of the invention includes derivatives of nucleoside-5′-O-hypophosphates and their mono- and dithiohypophosphate analogues, in particular 5′-O-[β,β-dialkyl-(α-thiohypophosphate)]- and 5′-O-[β,β-dialkyl-(α,α-dithiohypophosphate)]-and 5′-O-[β,β-dialkyl-(α,β-dithiohypophosphate)]- and 5′-O-[β-alkyl-(α-thiohypophosphate)]- and 5′-[β-alkyl-(α,α-dithiohypophosphate)]- and 5′-O-(α-thiohypophosphate)]- and 5′-O-(α,α-dithiohypophosphate-nucleosides of general formula 1,wherein A¹ is a fluorine atom, azide or hydroxyl group, A² is a hydrogen atom, B¹ is adenine, 2-chloroadenine, 2-bromoadenine, 2-fluoroadenine, 2-iodoadenine, hypoxantine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil, 2-pyrimidione residue, W¹ is an oxygen or carbon atom or a methylidene group, W² is a carbon atom or W² with A¹ and A² represent a sulfur atom or an oxygen atom, Z¹ is a hydrogen or fluorine atom or a hydroxyl group or an alkoxyl group, Z² is a hydrogen or fluorine atom or a hydroxyl or methyl group or Z¹ and Z² jointly represent a fluoromethylene group or A¹, A², Z¹ and Z² jointly represent a carbon-carbon double bond, X₁, X₂ and Y represent an oxygen atom or a sulfur atom, R¹ and R² represent an alkyl, aryl or a hydrogen atom associated with amine, and the process for the manufacture of derivatives of nucleoside-5′-O-hypophosphates and their mono- and dithiohypophosphate analogues of general formula 1,wherein A¹, A², B¹, W¹, W², Z¹, Z², R¹, R², X₁, X₂ and Y are as above.

Nucleoside polyphosphates whose structures contain a phosphorus-phosphorus bond between the phosphorus atoms at the alpha and beta positions of the polyphosphate chain may reveal inhibiting activity with respect to polymerases.

The derivatives of nucleoside-5′-O-hypophosphates and their mono- and dithiohypophosphate analogues, in particular 5′-O-[β,β-dialkyl-(α-thiohypophosphate)]- and 5′-O-[β,β-dialkyl-(α,α-dithiohypophosphate)]- and 5′-O-[β,β-dialkyl-(α,β-dithiohypophosphate)- 5′-O-[β-alkyl-(α-thiohypophosphate)]- and 5′-O-[β-alkyl-(β,β-dithiohypophosphate)]- and 5′-O-(α-thiohypophosphate)]- and 5′-O-(α,α-dithiohypophosphate)-nucleosides of the present invention are of general formula 1,wherein A¹ is a fluorine atom, azide or hydroxyl group, A² is a hydrogen atom, B¹ is adenine, 2-chloroadenine, 2-bromoadenine, 2-fluoroadenine, 2-iodoadenine, hypoxantine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil, 2-pyrimidione residue, W¹ is an oxygen or carbon atom or a methylidene group, W² is a carbon atom or W², A¹ and A² jointly represent a sulfur atom or an oxygen atom, Z¹ is a hydrogen or fluorine atom or a hydroxyl group or an alkoxyl group, Z² is a hydrogen or fluorine atom or a hydroxyl or methyl group or Z¹ and Z² jointly represent a fluoromethylene group or A¹, A², Z¹ and Z² jointly represent a carbon-carbon double bond, X₁, X² and Y represent an oxygen atom or a sulfur atom, R¹ and R² represent an alkyl, aryl or a hydrogen atom associated with amine.

The process for the manufacture of derivatives of nucleoside-5′-O-hypophosphates and their mono- and dithiohypophosphate analogues of general formula 1, wherein A¹, A², B¹, R¹, R², W¹, W², Z¹, Z², X₁, X₂ and Y are as above according to the present invention consists in that the nucleoside derivatives of general formula 2, wherein R³, R⁴, R⁵ and R⁶ represent a hydrogen atom, simple alkyl or aryl with 1 to 6 carbon atoms, wherein A², W¹ are as above, A³ is a fluorine atom, azide group or a protected hydroxyl group, W² is a carbon atom or A², A³, W² jointly represent a sulfur atom or oxygen atom, B² is adenine, 2-chloroadenine, 2-bromoadenine, 2-fluoroadenine, 2-iodoadenine, hypoxantine, guanine or cytosine residue of formulae 3, 4, 5 wherein Z⁵ is a hydrogen atom or a known exoamine protecting group, Z⁶ is a hydrogen atom or a chlorine, fluorine, bromine or iodine atom, Z⁷ is a hydrogen atom or a chlorine, fluorine, bromine or iodine atom or B² is a thymine residue or azacytosine residue or 5-fluorouracil, 5-bromouracil, 5-iodouracil, 5-chlorouracil, 5-(2-bromovinyl)uracil residue or 2-pyrimidione residue and Z³ is a hydrogen, fluorine atom or a protected hydroxyl group, Z⁴ is a hydrogen, fluorine atom or a protected hydroxyl group or a methyl group or Z³ and Z⁴ jointly represent a fluoromethyl group or A², A³, Z³, Z⁴ jointly represent a carbon-carbon double bond undergo a condensation reaction with phosphorous acid diesters of general formula (R⁷O)(R⁸O)POH or thiophosphorous acid diesters of general formula (R⁷O)(R⁸O)PSH, wherein R⁷ and R⁸ represent an alkyl or aryl, and the condensation is carried out in anhydrous organic solvents in the presence of condensation activators and after reaction completion the groups which protect 2′- and 3′-hydroxyl groups and the groups which protect nucleoside exoamine groups are removed according to known prior art.

The protective groups for the 2′- and 3′-hydroxyl groups preferably include known protecting groups selected from a group consisting of the acyl, benzoyl, 4,4′-dimethoxytriphenylmethyl, benzyl, trialkylsilyl, in particular a trimethylsilyl group.

The protective groups used for the exoamine groups include known protecting groups preferably selected from a group consisting of the phenoxyacetyl, isopropoxyacetyl, isobutyryl, benzoyl, (dialkylamino)methylidene and (dialkylamino)ethylidene group.

The condensation activators used include non-nucleophilic alcoholates, such as potassium tert-butanolate, or amines, such as imidazole, 1-methylimidazole, 4-dimethylaminopyridine, triethylamine and in particular 1,8-diazabicyclo[5.4]undec-7-ene (DBU).

The condensation reaction is preferably carried out in an anhydrous organic solvent selected from a group consisting of acetonitrile, methylene chloride, N,N-dimethylformamide, pyridine, dioxane and tetrahydrofuran.

In the process according to the present invention, compounds of formula 1,wherein X₁, X₂ and Y represent an oxygen atom, are preferably obtained from previously prepared compounds of formula 1 wherein X₁=S or X₁=O, X₂=S, Y=S or Y=O in the oxidation reaction using oxidation reagents known in the art, particularly iodosobenzene and iodoxobenzene. The process according to the present invention is general and may be used in the direct synthesis of nucleoside-5′-O-hypophosphates of general formula 1.

In the process according to the present invention, compounds of formula 1,wherein R¹ represents a hydrogen atom associated with amine, are preferably obtained from previously prepared compounds of formula 1,wherein R¹ is a methyl group and R² is an alkyl or aryl in the reaction with primary amines or ammonia, particularly with tert-butylamine. The process according to the present invention is general and may be used in the direct synthesis of 5′-O-[β-alkyl(α-thiohypophosphate)]- and 5-O-[β-alkyl-(α,α-dithiohypophosphate)]nucleosides of general formula 1.

In the process according to the present invention, compounds of formula 1,wherein R¹ and R² represent a hydrogen atom associated with amine, are preferably obtained from previously prepared compounds of formula 1, wherein R¹ and R² represent an alkyl or R¹ is a hydrogen atom associated with amine and R² is an alkyl in the reaction with trimethylsilyl halide, particularly with bromotrimethylsilane. The process according to the present invention is general and may be used in the direct synthesis of 5′-O-(α-thiohypophosphate)- and 5′-O-(α, α-dithiohypophosphate)-nucleosides of general formula 1.

The process of the invention may be utilised to manufacture 5′-O-[β,β-dialkyl-(α-thiohypophosphate)]- and 5′-O-[β,β-dialkyl-(α,α-dithiohypophosphate)]- and 5′-O-[β,β-dialkyl-(α,β-dithiohypophosphate)- and 5′-O-[β-alkyl-(α-thiohypophosphate)]- and 5′-O-[β-alkyl-(α,α-dithiohypophosphate)]- and 5′-O-(α-thiohypophosphate)]- and 5′-O-(α,α-dithiohypophosphate)-nucleosides of general formula 1,wherein A¹ is a fluorine atom, azide or hydroxyl group, A² is a hydrogen atom, B¹ is adenine, 2-chioroadenine, 2-bromoadenine, 2-fluoroadenine, 2-iodoadenine, hypoxantine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil, 2-pyrim idione residue, W¹ is an oxygen or carbon atom or a methylidene group, W² is a carbon atom or A¹, A², W² jointly represent a sulfur atom or an oxygen atom, Z¹ is a hydrogen or fluorine atom or a hydroxyl group or an alkoxyl group , Z² is a hydrogen or fluorine atom or a hydroxyl or methyl group or Z¹ and Z² jointly represent a fluoromethylene group or A¹, A², Z¹ and Z² jointly represent a carbon-carbon double bond, X¹, X² and Y represent an oxygen atom or a sulfur atom, and X¹, X² and Y may independently represent an oxygen or sulfur atom, R¹ and R² represent an alkyl, aryl or a hydrogen atom associated with amine.

The process according to the present invention is illustrated in the examples which follow.

### Example I

5′-O-[β,β-diethyl-(α-thiohypophosphate)]-uridine

To a solution of 0.05 mmol of 5′-(2-thio-[1,3,2]-oxathiaphospholanyl)-O^{2′}, O^{3′}-diisopropoxyacetyluridine in 0.5 mL of acetonitrile 0.05 mmol of diethyl phosphite was added and subsequently 0.055 mmol of DBU was added dropwise. The reaction was carried out at ambient temperature for 2.5 hours (TLC and ³¹P NMR analyses). The reaction mixture was then concentrated under reduced pressure and aqueous saturated ammonia (3 mL) was added to the residue (ambient temperature, 1 hour). The ammonia was subsequently distilled off under reduced pressure. The product was isolated in a 19% yield using ion-exchange chromatography (DEAE-Sephadex A-25) with TEAB (0.10-0.80 M; pH=7.5) as the eluent. ³¹P NMR(D₂O)δ:55.790, 13.225 ppm, ¹Jₚ₋ₚ=501 Hz, MALDI-TOF m/z:(M-1) 459.2.

### Example II

5′-O-[β,β-dimethyl-(α-thiohypophosphate)]-uridine

To a solution of 0.05 mmol of 5′-(2-thio-[1,3,2]-oxathiaphospholanyl)-O^{2′}, O^{3′}-diisopropoxyacetyluridine in 0.5 mL of acetonitrile 0.05 mmol of dimethyl phosphite was added and subsequently 0.055 mmol of DBU was added dropwise. The reaction was carried out at ambient temperature for 2.5 hours (TLC and ³¹P NMR analyses). The reaction mixture was then concentrated under reduced pressure and aqueous saturated ammonia (3 mL) was added to the residue (ambient temperature, 1 hour). The ammonia was subsequently distilled off under reduced pressure. The product was isolated in a 26% yield using ion-exchange chromatography (DEAE-Sephadex A-25) with TEAB (0.10-0.60 M; pH=7.5) as the eluent. ³¹P NMR(D₂O)δ:55.177, 15.653 ppm, ¹Jₚ₋ₚ=501 Hz, MALDI-TOF m/z:(M-1) 431.0.

### Example III

5′-O-[β-methyl-(α-thiohypophosphate)]-uridine

To 6 μmol of 5′-O-[β,β-dimethyl-(α-thiohypophosphate)]-uridine 0.5 ml of t-butylamine was added. The reaction was carried out at ambient temperature for 4 days (HPLC and ³¹P NMR analyses) until the complete conversion of the substrate into the product. The reaction mixture was subsequently concentrated under reduced pressure with the final yield of 100%. ³¹P NMR(D₂O)δ:65.107, 9.813 ppm, ¹Jₚ₋ₚ=531 Hz, MALDI-TOF m/z:(M-2) 416.9.

### Example IV

5′-O-α-thiohypophosphate)-uridine

To a solution of 0.05 mmol of 5′-(2-thio-[1,3,2]-oxathiaphospholanyl)-O², O³-diisopropoxyacetyluridine in 0.5 mL of acetonitrile 0.05 mmol of dimethyl phosphite was added and subsequently 0.055 mmol of DBU was added dropwise. The reaction was carried out at ambient temperature for 2.5 hours (TLC and ³¹P NMR analyses). The reaction mixture was subsequently cooled to −40° C. and 0.2 mmol of bromotrimethylsilane was added dropwise. The mixture was heated at a rate of 10° C. per 0.5 hour. Once the mixture was heated to ambient temperature, the reaction was carried out for 12 hours. The reaction mixture was then concentrated under reduced pressure and aqueous saturated ammonia (3 mL) was added to the residue (ambient temperature, 1 hour). The ammonia was subsequently distilled off under reduced pressure. The product was isolated in a 18% yield using ion-exchange chromatography (DEAE-Sephadex A-25) with TEAB (0.10-0.60 M; pH=7.5) as the eluent and gel filtration on Sephadex LH-20 using water as the eluent. ³¹P NMR(D₂O)δ:66.366, 7.643 ppm, ¹Jₚ₋ₚ=543 Hz, MALDI-TOF m/z:(M-1) 403.0.

### Example V

5′-O-[β,β-diethyl-(α,β-dithiohypophosphate)]-uridine

To a solution of 0.05 mmol of 5′-(2-thio-[1,3,2]-oxathiaphospholanyl)-O^{2′}, O^{3′}-diisopropoxyacetyluridine in 0.5 mL of acetonitrile 0.05 mmol of diethyl thiophosphite was added and subsequently 0.055 mmol of DBU was added dropwise. The reaction was carried out at ambient temperature for 16 hours (TLC and ³¹P NMR analyses). The reaction mixture was then concentrated under reduced pressure and aqueous saturated ammonia (3 mL) was added to the residue (ambient temperature, 1 hour). The ammonia was subsequently distilled off under reduced pressure. The product was isolated in a 23% yield using ion-exchange chromatography (DEAE-Sephadex A-25) with TEAB (0.10-0.60 M; pH=7.5) as the eluent. ³¹P NMR(D₂O)δ:83.995, 83.804, 60.632, 60.467 ppm, ¹Jₚ₋ₚ=384 Hz, MALDI-TOF m/z:(M-1) 475.1.

### Example VI

5′-O-β-methylhypophosphatecytidine

To a solution of 16 μmol of 5′-O-[β-methyl-(α-thiohypophosphate)]-cytidine in 2 ml of methanol 16 μmol of iodoxobenzene was added. The reaction was carried out at ambient temperature for 12 hours (HPLC and ³¹P NMR analyses). The reaction mixture was subsequently concentrated under reduced pressure. The product was isolated in an 82% yield using ion-exchange chromatography (DEAE-Sephadex A-25) with TEAB (0.0-0.3 M; pH=7.5) as the eluent. ³¹P NMR(D₂O)δ:9.725 ppm, MALDI-TOF m/z:(M-1) 412.0.

### Example VII

5′-O-β,β-dimethylhypophosphateuridine

To a solution of 15 μmol of 5′-O-[β,β-dimethyl-(α-thiohypophosphate)]-uridine in 0.5 ml of methanol 15 μmol of iodoxobenzene was added. The reaction was carried out at ambient temperature for 12 hours (HPLC and ³¹P NMR analyses). The reaction mixture was subsequently concentrated under reduced pressure. The product was isolated in a 79% yield using ion-exchange chromatography (DEAE-Sephadex A-25) with TEAB (0.0-0.3 M; pH=7.5) as the eluent. ³¹P NMR(D₂O)δ:19.488, −0.450 ppm, ¹Jₚ₋ₚ=657 Hz, MALDI-TOF m/z:(M-1) 415.0.

### Example VIII

5′-O-[β-methyl-(α-thiohypophosphate)]-2′-O-methyl-guanosine

To a solution of 0.20 mmol of 5′-(2-thio-[1,3,2]-oxathiaphospholanyl)-O-^{3′}-acety-2′-O-methyl-N-isobutyryl-guanosine in 1 mL of acetonitrile 0.20 mmol of dimethyl phosphite was added and subsequently 0.23 mmol of DBU was added dropwise. The reaction was carried out at ambient temperature for 2.5 hours (TLC and ³¹P NMR analyses). The reaction mixture was then concentrated under reduced pressure and aqueous saturated ammonia (3 mL) was added to the residue (temperature 45° C., 4 hour). The ammonia was subsequently distilled off under reduced pressure. The product was isolated in a 16% yield using ion-exchange chromatography (DEAE-Sephadex A-25) with TEAB (0.10-0.60 M; pH=7.5) as the eluent. ³¹P NMR(D₂O)δ:60.79, 6.28 ppm, ¹Jₚ₋ₚ=450 Hz, MALDI-TOF m/z:(M-1) 470.1.

1-9. (canceled)

10. Thio- and dithio- analogues of nucleoside-5′ -O-hypophosphates of general formula 1,wherein A¹ represents a fluorine atom or azide or hydroxyl group, A² represents a hydrogen atom, B¹ represents an adenine, 2-chloroadenine, 2-fluoroadenine, 2-bromoadenine, 2-iodoadenine, hypoxanthine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil or 2-pyrimidione residue, W¹ represents an oxygen or a methylene group, W² represents a carbon atom and A^{l} and A² represent a hydrogen or fluorine atom or azide or hydroxyl group, or A¹, A², W² jointly represent a sulfur or oxygen atom, Z¹ represents a hydrogen or fluorine atom or hydroxyl group or an alkoxyl group, Z² represents a hydrogen or fluorine atom or hydroxyl or methyl group or Z¹ and Z² jointly represent a fluoromethylene group, or A² and Z² jointly represent a carbon-carbon double bond and A¹ and Z¹ represent a hydrogen or fluorine atom or an alkoxyl group, X¹, X² and Y represent an oxygen or sulfur atom, R¹ and R² represent an alkyl or aryl or a hydrogen atom.

11. A process for the manufacture of thio- and dithio- analogues of nucleoside-5′-O-hypophosphates of general formula 1,wherein A¹ is a fluorine atom, azide or hydroxyl group, A² is a hydrogen atom, B¹ is adenine, 2-chloroadenine, 2-bromoadenine, 2-fluoroadenine, 2-iodoadenine, hypoxantine, guanine, cytosine, 5-fluorocytosine, 5-bromocytosine, 5-iodocytosine, 5-chlorocytosine, azacytosine, thymine, 5-fluorouracil, 5-bromouracil, 5-iodouracil, 5-chlorouracil, 5-(2-bromovinyl)uracil, 2-pyrimidione residue, W¹ is an oxygen or a methylene group, W² is a carbon atom and A¹ and A² represent a hydrogen or fluorine atom or azide or hydroxyl group, or A¹, A², W² represent a sulfur atom or an oxygen atom, Z¹ is a hydrogen or fluorine atom or a hydroxyl group or an alkoxyl group, Z² is a hydrogen or fluorine atom or a hydroxyl or methyl group or Z¹ and Z² jointly represent a fluoromethylene group or A² and Z² jointly represent a carbon-carbon double bond and A^{l} and Z¹ represent a hydrogen or fluorine atom or an alkoxyl group, X¹, X² and Y represent an oxygen atom or a sulfur atom, R¹ and R² represent an alkyl, aryl or a hydrogen atom associated with amine characterised in that the condensation involves phosphorous acid diesters of general formula (R⁷O)(R⁸O)POH or thiophosphorous acid diesters of general formula (R⁷O)(R⁸O)PSH, wherein R⁷ and R⁸ represent an alkyl or aryl with the nucleoside derivatives of general formula 2,wherein A², A³, B², R³, R⁴, R⁵, R⁶, W¹, W², Z³, Z⁴ are as above, X² and Y represent an oxygen atom or a sulfur atom, and the condensation is carried out in anhydrous organic solvents in the presence of condensation activators and after reaction completion the groups which protect 2′- and 3′-hydroxyl groups and the groups which protect nucleoside exoamine groups are removed according to known prior art.

12. Process according to claim 11 characterised in that the protective groups for 2′- and 3′-hydroxyl groups include known protecting groups selected from a group consisting of the acyl, aroyl, 4,4′-dimethoxytriphenylmethyl, arylalkyl, trialkylsilyl, and in particular trimethylsilyl group.

13. Process according to claim 11 characterised in that the protective groups used for exoamine groups include known protecting groups selected from a group consisting of the phenoxyacetyl, isopropoxyacetyl, isobutyryl, benzoyl, (dialkylamino)methylidene and (dialkylamino)ethylidene group.

14. Process according to claim 11 characterised in that the condensation activators used include non-nucleophilic alcoholates, such as potassium tert-butanolate, or amines, such as imidazole, 1-methylimidazole, 4-dimethylaminopyridine, triethylamine and in particular 1,8-diazabicyclo [5.4]undec-7-ene (DBU).

15. Process according to claim 11 characterised in that the condensation reaction is carried out in an anhydrous organic solvent selected from a group consisting of acetonitrile, methylene chloride, N,N-dimethylformamide, pyridine, dioxane and tetrahydrofuran.

16. Process according to claim 11 characterised in that a compound of formula 1,wherein X¹, X² and Y represent an oxygen atom, is obtained from previously prepared compounds of formula 1,wherein X¹=S or X¹=O, X²=S, Y=S or Y=O in an oxidation reaction using oxidation reagents known in the art, particularly iodosobenzene and iodoxobenzene.

17. Process according to claim 11 characterised in that a compound of formula 1,wherein R¹ represents a hydrogen atom associated with amine, is preferably obtained from previously prepared compounds of formula 1,wherein R¹ represents a methyl group and R² represents an alkyl or aryl in the reaction with primary amines or ammonia, particularly with tert-butylamine.

18. Process according to claim 11 characterised in that a compound of formula 1, wherein R¹ and R² represent positively charged counterion(s), is preferably obtained from previously prepared compounds of formula 1 wherein R¹ and R² represent an alkyl, or R¹ is a hydrogen atom or a positively charged counterion and R² is an alkyl, in the reaction with trimethylsilyl halide, particularly with bromotrimethylsilane.
